SEQUENCE LISTING (76 sequences)

SEQ ID NO: 1 - DNA, 22 bp, Artificial sequence
  CDKN2A genomic promoter fragment primer
aaagcagggggcactcatattc

SEQ ID NO: 2 - DNA, 20 bp, Artificial sequence
  CDK2NA genomic promoter fragment primer
tccgagcacttagcgaatgt

SEQ ID NO: 3 - DNA, 21 bp, Artificial sequence
  CDK2NA genomic promoter fragment primer
acgcactcaaacacgcctttg

SEQ ID NO: 4 - DNA, 21 bp, Artificial sequence
  CDK2NA genomic promoter fragment primer
caaacttcgtcctccagagtc

SEQ ID NO: 5 - DNA, 20 bp, Artificial sequence
  CDKN2A Exon 2 primer
ggaaattggaaactggaagc

SEQ ID NO: 6 - DNA, 19 bp, Artificial sequence
  CDKN2A Exon 2 primer
tctgagctttggaagctct

SEQ ID NO: 7 - DNA, 18 bp, Artificial sequence
  CDKN2A Exon 3 primer
gacggcaagagaggaggg

SEQ ID NO: 8 - DNA, 21 bp, Artificial sequence
  CDKN2A Exon 3 primer
aaaactacgaaagcggggtgg

SEQ ID NO: 9 - DNA, 18 bp, Artificial sequence
  CDKN2A RT-PCR promoter fragment primer
ttcaggggtgccacattc

SEQ ID NO: 10 - DNA, 17 bp, Artificial sequence
  CDKN2A RT-PCR promoter fragment primer
ccaggtccacgggcaga

SEQ ID NO: 11 - DNA, 17 bp, Artificial sequence
  RT-PCR 3UTR fragment primer
agcccaactgcgccgac

SEQ ID NO: 12 - DNA, 18 bp, Artificial sequence
  CDKN2A Exon 1 primer
gggagcagcatggagccg

SEQ ID NO: 13 - DNA, 18 bp, Artificial sequence
  CDKN2A Exon 1 primer
agtcgcccgccatcccct

SEQ ID NO: 14 - DNA, 19 bp, Artificial sequence
  CDKN2A Exon 1 primer
gaagaaagaggaggggctg

SEQ ID NO: 15 - DNA, 20 bp, Artificial sequence
  CDKN2A Exon 1 primer
gcgctacctgattccaattc

SEQ ID NO: 16 - DNA, 19 bp, Artificial sequence
  CDKN2A Exon 2 fragment primer
agcttcctttccgtcatgc

SEQ ID NO: 17 - DNA, 17 bp, Artificial sequence
  CDKN2A Exon 2 fragment primer
gcagcaccaccagcgtg

SEQ ID NO: 18 - DNA, 16 bp, Artificial sequence
  CDKN2A Exon 2 fragment primer
tggacgtgcgcgatgc

SEQ ID NO: 19 - DNA, 23 bp, Artificial sequence
  CDKN2A Exon 2 fragment primer
ggaagctctcagggtacaaattc

SEQ ID NO: 20 - DNA, 20 bp, Artificial sequence
  CDKN2A Exon 3 primer
ccggtagggacggcaagaga

SEQ ID NO: 21 - DNA, 25 bp, Artificial sequence
  CDKN2A Exon 3 primer
ctgtaggaccttcggtgactgatga

SEQ ID NO: 22 - DNA, 17 bp, Artificial sequence
  CDKN2A exon 1 probe
ccggggtcgggtagagg

SEQ ID NO: 23 - DNA, 17 bp, Artificial sequence
  CDKN2A Exon 1 mutant probe
ccggggtccggtagagg

SEQ ID NO: 24 - DNA, 16 bp, Artificial sequence
  CDKN2A Exon 1 probe
ggctgctggaggcggt

SEQ ID NO: 25 - DNA, 16 bp, Artificial sequence
  CDKN2A exon 1 mutant probe
ggctgccggaggcggt

SEQ ID NO: 26 - DNA, 18 bp, Artificial sequence
  CDKN2A Exon 2 probe
aggtcatgatgatgggca

SEQ ID NO: 27 - DNA, 18 bp, Artificial sequence
  CDKN2A Exon 2 mutant probe
aggtcatgatcatgggca

SEQ ID NO: 28 - DNA, 17 bp, Artificial sequence
  CDKN2A Exon 2 probe
accgggccggggcgcgg

SEQ ID NO: 29 - DNA, 17 bp, Artificial sequence
  CDKN2A Exon 2 mutant probe
accgggcctgggcgcgg

SEQ ID NO: 30 - DNA, 17 bp, Artificial sequence
  CDKN2A Exon 2 probe
tcgcgatgtcgcacggt

SEQ ID NO: 31 - DNA, 17 bp, Artificial sequence
  CDKN2A Exon 2 mutant probe
tcgcgatgacgcacggt

SEQ ID NO: 32 - DNA, 17 bp, Artificial sequence
  CDKN2A Exon 2 probe
tagatgccgcggaaggt

SEQ ID NO: 33 - DNA, 17 bp, Artificial sequence
  CDKN2A Exon 2 polymorphic probe
tagatgccacggaaggt

SEQ ID NO: 34 - DNA, 19 bp, Artificial sequence
  CDKN2A Exon 3 wildtype probe
gaaacctccggaaacttag

SEQ ID NO: 35 - DNA, 19 bp, Artificial sequence
  CDKN2A Exon 3 polymorphic probe
gaaacctcgggaaacttag

SEQ ID NO: 36 - DNA, 17 bp, Artificial sequence
  CDKN2A Exon 3 wildtype probe
tacagggccacaactgg

SEQ ID NO: 37 - DNA, 17 bp, Artificial sequence
  CDKN2A Exon 3 polymorphic probe
tacagggctacaactgg

SEQ ID NO: 38 - DNA, 21 bp, Artificial sequence
  CDKN2A sequencing primer 427F
tgccccagacagccgttttac

SEQ ID NO: 39 - DNA, 20 bp, Artificial sequence
  hMSHR N-outer primer
agatggaaggaggcaggcat

SEQ ID NO: 40 - DNA, 26 bp, Artificial sequence
  hMSHR C-outer primer
ccgcgcttcaacactttcagagatca

SEQ ID NO: 41 - DNA, 21 bp, Artificial sequence
  hMSHR N-inner
cccctggcagcaccatgaact

SEQ ID NO: 42 - DNA, 21 bp, Artificial sequence
  hMSHR C-inner
tgcccagggtcacacaggaac

SEQ ID NO: 43 - DNA, 18 bp, Artificial sequence
  hMSHR N-inner sequencing primer 1
tctgacgggctcttcctc

SEQ ID NO: 44 - DNA, 18 bp, Artificial sequence
  hMSHR N-inner sequencing primer 3
tccagcctctgcttcctg

SEQ ID NO: 45 - DNA, 18 bp, Artificial sequence
  hMSHR N-inner sequencing primer 4
gcccggctccacaagagg

SEQ ID NO: 46 - DNA, 18 bp, Artificial sequence
  hMSHR C-inner sequencing primer 5
gcgctgcctcttgtggag

SEQ ID NO: 47 - DNA, 18 bp, Artificial sequence
  hMSHR C-inner sequencing primer 2
atggagctgcaggtgatc

SEQ ID NO: 48 - DNA, 20 bp, Artificial sequence
  MCIR forward PCR primer
caacgactccttcctgcttc

SEQ ID NO: 49 - DNA, 19 bp, Artificial sequence
  MC1R reverse PCR primer
tgcccagcacacttaaagc

SEQ ID NO: 50 - DNA, 20 bp, Artificial sequence
  MC1R internal forward sequencing primer
acctgcagctccatgctgtc

SEQ ID NO: 51 - DNA, 19 bp, MC1R reverse inner sequencing primer
gtcacgatgctgtggtagc

SEQ ID NO: 52 - DNA, 15 bp, Artificial sequence
  V60L Val probe
gctggtggtggccac

SEQ ID NO: 53 - DNA, 15 bp, Artificial sequence
  V60L Leu probe
gctggtgttggccac

SEQ ID NO: 54 - DNA, 15 bp, Artificial sequence
  K65N Lys probe
tcgccaagaaccgga

SEQ ID NO: 55 - DNA, 15 bp, Artificial sequence
  K65N Asn probe
tcgccaataaccgga

SEQ ID NO: 56 - DNA, 15 bp, Artificial sequence
  D84E Asp probe
tgtcggacctgctgg

SEQ ID NO: 57 - DNA, 15 bp, Artificial sequence
  D84E Glu probe
tgtcggaactgctgg

SEQ ID NO: 58 - DNA, 15 bp, Artificial sequence
  V92M Val probe
gagcaacgtgctgga

SEQ ID NO: 59 - DNA, 15 bp, Artificial sequence
  V92M Met probe
gagcaacatgctgga

SEQ ID NO: 60 - DNA, 15 bp, Artificial sequence
  V92L Leu probe
gagcaacttgctgga

SEQ ID NO: 61 - DNA, 15 bp, Artificial sequence
  R142H Arg probe
gtggaccgctacatc

SEQ ID NO: 62 - DNA, 15 bp, R142H His
gtggaccactacatc

SEQ ID NO: 63 - DNA, 15 bp, Artificial sequence
  R151C Arg probe
cgcactgcgctacca

SEQ ID NO: 64 - DNA, 15 bp, Artificial sequence
  R151C Cys probe
cgcactgtgctacca

SEQ ID NO: 65 - DNA, 15 bp, Artificial sequence
  I55L Ile probe
cacagcatcgtgacc

SEQ ID NO: 66 - DNA, 15 bp, Artificial sequence
  I155L Leu probe
cacagcaccgtgacc

SEQ ID NO: 67 - DNA, 15 bp, Artificial sequence
  R160W Arg probe
cctgccgcgggcgcg

SEQ ID NO: 68 - DNA, 15 bp, Artificial sequence
  R160W Trp probe
cctgccgtgggcgcg

SEQ ID NO: 69 - DNA, 15 bp, Artificial sequence
  R163Q Arg probe
gcgcggcgagccgtt

SEQ ID NO: 70 - DNA, 15 bp, Artificial sequence
  R163Q Gln probe
gcgcggcaagccgtt

SEQ ID NO: 71 - DNA, 15 bp, Artificial sequence
  D294H Asp probe
catcatcgaccccct

SEQ ID NO: 72 - DNA, 15 bp, Artificial sequence
  D294H His probe
catcatccaccccct

SEQ ID NO: 73 - DNA, 15 bp, Artificial sequence
  A299T Ala probe
catctacgccttcca

SEQ ID NO: 74 - DNA, 15 bp, Artificial sequence
  A299T Thr probe
catctacaccttcca

SEQ ID NO: 75 - DNA, 15 bp, Artificial sequence
  537insC wild-type probe
gctcttcatcgccta

SEQ ID NO: 76 - DNA, 15 bp, Artificial sequence
  537insC insC probe
gctcttccatcgcct

(19) United States
(12) Patent Application Publication
(10) Pub. No.: US 2003/0175721 A1
(43) Pub. Date: Sep. 18, 2003

(54) Melanoma risk detection

(76) Inventors: Neil F. Box, Queensland (AU); David L. Duffy, Stafford (AU); Nicholas K. Hayward, Queensland (AU); Nicholas G. Martin, Queensland (AU); Richard A. Sturm, Queensland (AU); Nelleke A. Gruis, Leiderdorp (NL); Pieter A. Van Der Velden, Leiden (NL); Wilma Bergman, Oegstgeest (NL); Rune R. Frants, Hoofddorp (NL)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/108,732

(22) Filed: Mar. 28, 2002

Related U.S. Application Data
(60) Provisional application No. 60/279,515, filed on Mar. 28, 2001.

(51) Int. Cl. C12Q 1/68

(52) U.S. Cl. 435/6

(57) ABSTRACT

A method of determining predisposition to melanoma is provided in the form of a molecular diagnostic method that detects the presence of a cyclin dependent kinase inhibitor 2A mutant allele and/or a melanocortin-1 receptor variant allele. The presence of a cyclin dependent kinase inhibitor 2A mutant allele increases the probability that an individual carrying a melanocortin-1 receptor variant allele will develop melanoma. Similarly, the presence of a melanocortin-1 receptor variant allele increases the probability that an individual carrying a cyclin dependent kinase inhibitor 2A mutant allele will develop melanoma.

### CROSS-REFERNCE TO RELATED APPLICATIONS

This application is entitled to priority pursuant to 35 U.S.C. § 119(e) to U.S. provisional patent application 60/279,515, which was filed on Mar. 28, 2001.

### BACKGROUND OF THE INVENTION

Germline mutations in three different genes have been shown to influence risk of melanoma, specifically those encoding: cyclin dependent kinase inhibitor 2A (CDKN2A) (Hussussian et al. 1994; Kamb et al. 1994a), cyclin dependent kinase 4 (CDK4) (Zuo et al. 1996; Soufir et al. 1998) and the melanocortin-1 receptor (MC1R) (Palmer et al. 2000). The basis of melanoma risk as determined by these genes probably revolves around at least two independent pathways (Whiteman et al. 1998), one being the CDKN2A/CDK4 cell cycle and tumor suppressor gene axis, and the other a pigmentary mediated predisposition axis implicated by the recent association of MC1R variants with red hair, fair skin, freckling and melanoma (Palmer et al. 2000). Melanoma risk attributable to MC1R may arise through the action of solar UV-light on lighter skin tones with diminished tanning capacity (Bliss et al. 1995; Breitbart et al. 1997) or possibly through a more direct intrinsic effect on melanocytic cellular transformation. On the other hand, linkage of a quantitative trail locus (QTL) accounting for 33% of variance in flat mole count to the 9p21-22 region containing CDKN2A (Zhu et al. 1999) suggests that CDKN2A mutation may play a role in determination of mole density which in turn predisposes to later melanoma formation; this appears to be a risk factor quite distinct from the red hair and fair skin associated with MC1R variants (Garbe et al. 1994; Bliss et al. 1995; Grange et al. 1995; Grulich et al. 1996).

The p16^{INK4A} protein encoded by the CDKN2A locus (Kamb et al. 1994b; Nobori et al. 1994) acts as a tumor suppressor that induces G1 cell cycle arrest through binding to and inhibiting the kinase activities associated with cyclin D complexes with both cyclin-dependent kinase 4 and 6 (CDK4 and CDK6). G1-S phase transition is usually dependent upon phosphorylation of the retinoblastoma protein (pRB) by the cyclinD1/CDK4 complex (Serrano et al. 1993; Lukas et al. 1995). Inactivation of CDKN2A via one of several mechanisms (homozygous deletion, mutation and/or promoter methylation) is a frequent event in tumors of many types (e.g. Kamb et al. 1994b; Nobori et al. 1994; reviewed by Ruas and Peters 1998). Furthermore, presence of mutations of this locus in the germline of a small proportion of familial melanoma patients (e.g. Hussussian et al. 1994; Kamb et al. 1994a; reviewed by Hayward 1998) indicates that p16 inactivation is an early and possibly initiating step in melanoma tumorigenesis.

MC1R is a seven-pass transmembrane G-protein-coupled receptor expressed in skin melanocytes that activates adenylate cyclase to elevate cyclic adenosine monophosphate levels upon stimulation by the proopiomelanocortin-derived peptides α-melanocyte stimulating hormone (α-MSH) and adrenocorticotropic hormone (Thody and Graham 1998; Suzuki et al 1996; Tsatmalia et al. 1999a; Tsatmalia et al. 1999b). Hornonal stimulation of MC1R leads to eumelanogenesis and is central to the tanning response of human skin following UV irradiation (Suzuki et al. 1999). During UV-exposure of the skin there is an increase in melanin production which may occur in part via an increase in tyrosinase gene transcription and enzyme activity (Gilchrest et al. 1996; Sturm et al. 1998). MC1R also regulates the balance of two distinct melanin types, the red/yellow pheomelanin and black/brown eumelanin, which in turn forms the basis of the association between specific MC1R alleles and red hair and fair skin (Valverde et al. 1995; Box et al. 1997; Smith et al. 1998; Flanagan et al. 2000; Palmer et al. 2000). It has already been shown that three common MC1R variant alleles, Arg151Cys, Arg160Tip and Asp294His are associated with an increased risk of melanoma within the Queensland population mediated at least in part by an effect on pigmentation phenotype (Palmer et al. 2000).

Germline mutations in CDKN2A have been identified in approximately 40% of the 5-10% of melanoma cases that occur in multiplex families. A recent population-based assessment of the overall caseload of melanoma in Queensland estimated that around 0.2% of all melanomas may be attributed to germline mutation of this gene (Aitken et al. 1999). Mutations within the CDK4 gene are even less frequent than within CDKN2A (Hayward 1999). On the other hand, MC1R variants have been identified at much higher frequency but with much lower genotype relative risk for melanoma (Box et al. 1997; Smith et al. 1998; Palmer et al. 2000; Healy et al. 1999). Nevertheless, because of the high frequency of MC1R variants in melanoma cases (Palmer et al. 2000) it has been estimated that as many as one third of melanomas in Queensland may be attributable to MC1R genotype.

### BRIEF SUMMARY OF THE INVENTION

The present inventors have realized that although there are several genetic risk factors that contribute to the likelihood of suffering from melanoma, there is little if any data that address the relative influence of genetic risk factors and interactions between underlying genes.

It is therefore an object of the invention to provide an improved method of determining whether an individual is predisposed to melanoma.

In one aspect, the invention provides a method of identifying a predisposition to melanoma, said method including the step of determining whether an individual carrying a CDKN2A mutant allele also carries an MC1R variant allele, a presence of said variant MC1R allele indicating a predisposition of said individual to melanoma greater than that expected as a consequence of said CDKN2A mutant allele alone.

In another aspect, the invention provides a method of identifying a predisposition to melanoma, said method including the step of determining whether an individual carrying a MC1R variant allele also carries a CDKN2A mutant allele, a presence of said CDKN2A mutant allele indicating a predisposition of said individual to melanoma greater than that expected as a consequence of said MC1R variant allele alone.

In yet another aspect, the invention provides a method of identifying a predisposition of to melanoma, said method including the step of determining whether an individual carries an MC1R variant allele and a CDKN2A mutant allele, a presence of said CDKN2A mutant allele and said MC1R variant allele indicating a predisposition of said individual to melanoma greater than that expected as a consequence of said CDKN2A mutant allele or said MC1R variant allele alone.

Preferably, the CDKN2A mutant allele is selected from the group consisting of: Gln50Arg; Arg24Pro; 46delC; Leu32Pro; Asp108Asn; Leu16Pro; Gly35Ala; 9del24; 33ins24; p16-Leiden and Met53Ile.

Preferably, the MC1R variant allele is selected from the group consisting of: Val60Leu; Asp84Glu; Val92Met; Arg142His; Arg151Cys; Ile155Thr; Arg160Trp; Arg163Gln; and Asp294His.

More preferably, the MC1R variant allele is selected from the group consisting of: Arg151Cys; Arg160Trp; and Asp294His.

In a particular embodiment, when the MC1R variant allele is Arg151Cys the CDKN2A mutant allele is not p16-Leiden.

In a further aspect, the invention provides a kit for identifying a predisposition to melanoma, said kit comprising one or more MC1R gene-specific primers and/or one or more CDKN2A gene-specific primers. The kit may further comprise one or more MC1R variant allele-specific probes and/or one or more CDKN2A mutant allele-specific probes.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE SEVERAL VIEWS OF THE DRAWINGS

FIG. 1. Smoothed survival curves (Cox proportional hazards model) for cutaneous malignant melanoma versus CDKN2A and MC1R genotypes. Smoothing was performed using the shape of the densely populated “CDKN2A mutation, MC1R variant” curve to infer the shape of the other 3 genotype covariate curves.

FIG. 2. Example of the inheritance of p16-Leiden and MC1R variant Arg151Cys in a Dutch FAMMM kindred. Grey symbols are melanoma cases and open symbols are unaffected individuals at the time of this study. Carriers of a 19 base pair (bp) deletion in the p16 gene (p16-Leiden) are indicated by del. Arg151Cys carriers are indicated by a +. The a priori probability to inherit the Arg151Cys variant is shown underneath the family members. In Dutch FAMMM, the Arg151Cys variant segregates more often with melanoma than expected (p≈0.07).

### DETAILED DESCRIPTION OF THE INVENTION

This invention relates to melanoma risk assessment. In particular, this invention relates to a molecular genetic method for determining whether an individual is predisposed to melanoma. More particularly, this invention relates to a method for determining melanoma risk according to the presence of a cyclin dependent kinase inhibitor 2A mutant allele and a melanocortin-1 receptor variant allele.

The present invention is predicated, at least in part, on the unexpected discovery that MC1R genotype impacts upon penetrance of CDKN2A mutations with respect to an individual's predisposition to melanoma. As will be described in more detail hereinafter, each particular CDKN2A mutation surveyed appears to act similarly with respect to melanoma risk. Importantly, however, MC1R variant alleles Arg151Cys; Arg160Trp; and Asp294His disproportionately contribute to an increase in melanoma risk in individuals carrying a CDKN2A mutation. This discovery has led to the realization that diagnostic methods may be established whereby melanoma risk can be assessed more accurately, based on the presence of a CDKN2A mutation and the presence of an MC1R variant allele, preferably selected from Arg151Cys; Arg160Trp; and Asp294His. Such methods may be useful in “stand alone” melanoma risk assessments such as in genetic screening and counselling applications, or in assisting and confirming diagnoses obtained by other genetic tests or more traditional symptom-based diagnosis.

As used herein, “predisposed” means having an increased probability that said individual may display or develop symptoms of melanoma relative to the population at large.

The present invention demonstrates increased “penetrance” of CDKN2A mutations when an individual also carries one or more copies of an MC1R variant allele (i.e homozygous or heterozygous with respect to the MC1R variant allele). In this context, by “penetrance” is meant the probability that an individual will develop a disease (in this case, melanoma) given that the individual has a particular genotype (in this case, a CDKN2A mutant allele and an MC1R variant allele).

Suitably, said individual is a human.

In the first-mentioned aspect of the invention, the CDKN2A mutant genotype of the individual is already known. MC1R genotype is determined to ascertain whether the individual has an increased melanoma predisposition compared to that resulting from the CDKN2A mutant genotype.

In the second-mentioned aspect of the invention, the MC1R variant genotype of the individual is known, and CDKN2A genotype is determined. The presence of a CDKN2A mutant allele indicates that the individual has an increased melanoma predisposition compared to that resulting from the MC1R variant genotype.

In the third-mentioned aspect, the MC1R and CDKN2A genotype of the individual is unknown. The presence of a CDKN2A mutant allele and a MC1R variant allele indicates that the individual has an increased melanoma predisposition compared to that resulting from either the CDKN2A mutant genotype or the MC1R variant genotype.

Generally, the individual will be heterozygous with respect to the CDKN2A mutant allele, as there are very few known homozygous mutants. All of the individuals studied in Example 1 are heterozygotes. However, in Example 2, two of the individuals are homozygotes. Either way, homozygosity/heterozygosity does not appear to be a crucial risk factor.

Similarly, homozygosity/heterozygosity with respect to the MC1R variant allele detected is not a crucial factor in determining melanoma risk according to the method of the invention.

By “MC1R variant allele” is meant any allelic form of a gene encoding the human melanocortin type 1 receptor, or a fragment thereof, other than an allele encoding wild-type melanocortin type 1 receptor. Examples of a wild-type MC1R allele sequence are provided in Box et al. 1997, Rana et al. 1999 and Harding et al. 2000.

Examples of MC1R variant alleles are Val60Leu; Asp84Glu; Val92Met; Arg142His; Arg151Cys; Ile155Thr; Arg160Trp; Arg163Gln; and Asp294His, although the term “MC1R variant allele” is not limited to these.

By “CDKN2A mutant allele” is meant any allelic form of a CDKN2A gene that has a nucleotide sequence variation compared to wild-type, wherein the sequence variation affects the level of expression or the function of the CDKN2A gene or its protein product. The types of sequence variations contemplated include, but are not limited to, sequence deletions, sequence insertions, missense mutations, nonsense mutations, non-synonymous sequence variations, and sequence variations resulting from chromosomal rearrangement or deletion events.

Specific examples of CDKN2A mutant alleles are Gln50Arg; Arg24Pro; 46delC; Leu32Pro; Asp108Asn; Leu16Pro; Gly35Ala; 9del24; 33ins24; Met53Ile; and 225del19 otherwise known as p16-Leiden, although the term “CDKN2A mutant allele” is not limited to these.

Examples of CDKN2A mutant alleles and primer and probes useful in detecting said mutant alleles can be found in Walker et al. 1995, Castellano et al. 1997, Whiteman et al. 1997 and Pollock et al. 2001.

Suitably, the MC1R allele and/or the CDKN2A allele is isolated in a nucleic acid sample obtained from any suitable cells or tissues of a human. Examples of cells or tissues contemplated by the present invention include blood leukocytes, skin, buccal swabs, hair root sheath, semen or the like.

Preferably, said cells are blood leukocytes.

As used herein, “nucleic acid” encompasses single- or double-stranded mRNA, RNA, cRNA and DNA inclusive of cDNA and genomic DNA. Hence, “alleles” and “genes” according to the invention may be in any of the nucleic acid forms encompassed by this definition.

Preferably, said nucleic acid is genomic DNA, although RNA is also contemplated. In such a case, cDNA is reverse transcribed from RNA by methods well known in the art.

A preferred genomic DNA extraction technique, based on that of Miller et al. 1988, is described in detail hereinafter.

A “polynucleotide” is a nucleic acid having eighty (80) or more contiguous nucleotides, while an “oligonucleotide” has up to eighty (80) contiguous nucleotides.

A “probe” may be a single or double-stranded oligonucleotide or polynucleotide, suitably labeled for the purpose of detecting complementary sequences in Northern or Southern hybridization (such as ³²p or digoxigenin labeling, for example).

Examples of CDKN2A allele-specific probes useful according to the present invention are provided in Table 3 (SEQ ID NOS: 22-37).

Examples of MC1R allele-specific probes useful according to the present invention are provided in Table 4 (SEQ ID NOS: 52-76).

A “primer” is usually a single-stranded oligonucleotide, preferably having 15-50 contiguous nucleotides, which is capable of annealing to a complementary nucleic acid template and being extended in a template-dependent fashion by the action of a DNA polymerase such as Taq polymerase, RNA-dependent DNA polymerase or Sequenase™.

Examples of CDKN2A gene-specific primers useful according to the present invention are provided in Tables 1 and 2 (SEQ ID NOS: 1-21).

Examples of MC1R gene-specific primers useful according to the present invention may be are selected from the group consisting of: SEQ ID NO: 39; SEQ ID NO: 40; SEQ ID NO: 41; SEQ ID NO: 42; SEQ ID NO: 48 and SEQ ID NO: 49.

The terms “anneal”, “hybridize” and “hybridization” are used herein in relation to the formation of bimolecular complexes by base-pairing between complementary or partly-complementary nucleic acids in the sense commonly understood in the art. It should also be understood that these terms encompass base-pairing between modified purines and pyrimidines (for example, inosine, methylinosine and methyladenosine) and modified pyrimidines (for example thiouridine and methylcytosine) as well as between A,G,C,T and U purines and pyrimidines. Factors that influence hybridization such as temperature, ionic strength, duration and denaturing agents are well understood in the art, although a useful operational discussion of hybridization is provided in to Chapter 2 of CURRENT PROTOCOLS IN MOLECULAR BIOLOGY (Eds. Ausubel et al. John Wiley & Sons NY, 2000), particularly at sections 2.9 and 2.10.

The method of the invention may be performed using any of a number of techniques useful in detecting nucleic acids of interest, in this case MC1R variant allele(s) and CDKN2A mutant allele(s).

Preferably, detection of MC1R and CDKN2A alleles includes a PCR amplification step using MC1R gene-specific and CDKN2A gene-specific primers respectively.

A particular PCR method that may also be useful is Bi-PASA (Bidirectional PCR Amplification of Specific Alleles), as for example described in Liu et al. 1997.

It will also be appreciated that nucleic acid sequence amplification techniques other than PCR may be useful according to the invention. Suitable nucleic acid amplification techniques other than PCR are well known to the skilled addressee and include strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994; ligase chain reaction (LCR) as for example described in International Application WO89/09385; and Q-β replicase amplification as for example described by Tyagi et al. 1996.

As used herein, an “amplification product” refers to a nucleic acid product generated by any nucleic acid amplification technique.

It will also be well understood by the skilled person that identification of MC1R and CDKNA alleles may be performed using any of a variety of techniques such as PCR-RFLP analysis, fluorescence-based melt curve analysis, nucleic acid arrays (e.g. microarrays), SSCP analysis, denaturing gradient gel electrophoresis (DGGE), ASO or direct sequencing of amplification products.

In preferred embodiments, MC1R and CDKNA alleles are identified by allele-ASO, SSCP analysis or sequencing of amplification products.

With regard to other embodiments, melt curve analysis can be performed using fluorochrome-labeled allele-specific probes which form base-pair mismatches when annealing to wild-type DNA strands in heterozygotes. Alternatively, fluorescent DNA-intercalating dyes can reveal the presence of these base-pair mismatches by virtue of their lower melting temperature (Tₘ) compared to fully complementary sequences. A useful example of allele-specific melt curve analysis can be found, for example, in International Publication No. WO97/46714.

Microarrays also utilize hybridization-based technology that, for example, may allow allele detection by way of hybridization of a nucleic acid sample to MC1R variant- and CDKN2A mutant-allele-specific probes immobilized on an appropriate substrate as is well understood in the art. In this regard, the skilled person is referred to Chapter 22 of CURRENT PROTOCOLS IN MOLECULAR BIOLOGY (Eds. Ausubel et al. John Wiley & Sons NY, 2000), International Publication WO00/58516, U.S. Pat. No. 5,677,195 and U.S. Pat. No. 5,445,934 which provide exemplary methods relating to nucleic acid array construction and use in detection of nucleic acids of interest.

DGGE also exploits Tₘ differences, but uses differential electrophoretic migration through gradient gels as a means of distinguishing subtle nucleotide sequence differences between alleles. Examples of DGGE methods can be found in Fodde & Losekoot, 1994, and U.S. Pat. Nos. 5,045,450 and 5,190,856.

So that the invention may be more readily understood and put into practical effect, the skilled person is directed to the following non-limiting examples, wherein Example 1 describes the influence of MC1R genotype upon individuals carrying any of a number of different CDKN2A gene mutations and Example 2 describes the influence of MC1R genotype upon p16-Leiden carriers with respect to melanoma risk.

### EXAMPLE 1

Patient Samples and DNA Extraction

The fifteen melanoma pedigrees available to this analysis were identified as part of the Queensland Familial Melanoma Project (Aitken et al. 1996). Briefly, strength of family history was determined in a total of 1,897 families ascertained as a subset of the 12,006 incident cases of histologically confirmed cutaneous melanoma diagnosed in residents of Queensland during the period from 1982 through 1990 and reported to the Queensland Cancer Registry. The previously described standardized family risk index (Aitken et al. 1996; Aitken et al. 1999) was used to divide the total sample of 1,897 families into three strata of familial melanoma risk, including 1,392 low, 414 intermediate, and 91 high risk families. Blood was collected and DNA extracted from members of selected families, PCR amplified and mutations detected by ASO hybridization or SSCP analysis, both as described previously (Aitken et al. 1999).

CDKN2A mutations were also detected by a method described in Castellano et al. 1997 and Pollock et al. 2001. Briefly, the promoter region was amplified using primers 96F and 968 R, the 5′UTR and exon 1 were amplified using primers and 781F and 1424R, exon 2 was amplified with primers 42F and 551R, and exon 3 was amplified with primers X3P2F and X3P2R (Pollock et al. 2001). All PCRs involved a ‘touchdown’ thermal cycling routine of 2 cycles at each annealing temperature, decreasing by 2 degrees followed by 25 cycles at the lower temperature. Each cycle consisted of 45 s at 94° C., 90 s at the annealing temperature and 90 s at 72° C. A 12 min initial denaturing step at 94° C. and a 3 min final extension step at 72° C. were also employed. PCR reactions consisted of 100-200 ng DNA, 10-30 pmol of each primer, 200 nM dNTPs (Promega, Madison, USA) and were performed with 1.25U of Amplitaq Gold (Hoffman-La Roche, Basel, Switzerland) in 100 mM Tris-HCl, pH 8.3, 500 mM KCl and 1.5 mM MgCl₂. Due to the high GC content of the CDKN2A gene, reactions were performed in a final concentration of 500 mM Betaine (Sigma Chemical Company, St Louis, USA). All products were electrophoresed on a 1.5% TAE agarose gel, excised and purified using a QIAquick Gel Extraction Kit (Qiagen, Hilden, Germany). The fragments were sequenced with the same primers used for the PCR step, and the 5′UTR was additionally sequenced with primer 427F 5′-TGCCCCAGACAGCCGTTTTAC-3′ (SEQ ID NO: 38; Pollock, 2001) using Applied Biosystems Incorporated (ABI) dye terminator sequencing kits according to the manufacturer's specifications. Sequencing products were run on an ABI 377 automated sequencer (PE Applied Biosystems, Foster City, USA).

Table 1 sets forth examples of PCR primer combinations useful in amplifying fragments of the CDKN2A gene for subsequent ASO analysis.

Table 2 sets forth examples of PCR prime combinations useful in amplifying generally shorter fragments of the CDKN2A gene for subsequent SSCP analysis.

Table 3 sets forth examples of oligonucleotide probes useful in ASO analysis of CDKN2A amplification products.

Extensive genotyping for CDKN2A germline mutations has resulted in identification of a total of 15 kindreds, all identified as high risk families, who contain a total of 9 different CDKN2A mutations Gln50Arg, Arg24Pro, 46delC, Leu32Pro, Asp108Asn, Leu16Pro, Gly35Ala, 9del24, 33ins24, or Met53Ile (Walker et al. 1995; Flores et al. 1997; Whiteman et al. 1997; Aitken et al. 1999).

An ongoing part of the family ascertainment procedure involved submission of questionnaires about standard melanoma risk factors, including propensity to burn in the sun, pigmentation (skin color at 21 years of age, and eye color), total freckling in summer, and density of melanocytic nevi. Data were also collected on melanoma age of onset, number of primary tumors, other tumors of non-melanocytic origin, and ancestry.

MC1R Genotyping

In order to obtain sufficient DNA product for sequencing and ASO dot blot detection of 9 previously identified MC1R variants including Val60Leu, Asp84Glu, Val92Met, Arg142His, Arg151Cys, Ile155Thr, Arg160Trp, Arg163Gln, and Asp294His, a nested primer PCR strategy was used for amplification of extracted genomic DNA from the melanoma families as previously described (Box et al. 1997). A nested primer PCR strategy was used to obtain sufficient DNA from the MC1R locus for direct sequence analysis and to allow ASO identification of haplotypes. The primers for amplification of the MC1R coding region exon were based on the nucleotide sequence reported by Mountjoy et al. 1992 (Accession number X65634). The first primer set hMSHR N-outer 5′-AGATGGAAGGAGGCAGGCAT-3′ (SEQ ID NO: 39) and C-outer 5′-CCGCGCTTCAACACTTTCAGAGATCA-3′ (SEQ ID NO: 40) are used in a 25 μl Taq DNA polymerase amplification reaction, containing 25 ng of genomic DNA, 10% DMSO initially denatured for 3 min at 94° C., followed by 30 cycles of 1 min 94° C., 1 min 55° C. and 3 min 72° C. ending with a 7 min 72° C. extension. Internal primers hMSHR N-inner 5′-CCCCTGGCAGCACCATGAACT-3′ (SEQ ID NO: 41) and C-inner 5′-TGCCCAGGGTCACACAGGAAC-3′ (SEQ ID NO: 42) are used in a second 25-50 μl reaction seeded using 5 μl of the first round reaction as template. Amplification conditions were identical to the first round.

DNA products from the Taq DNA polymerase amplification reactions are purified by agarose gel electrophoresis, isolated by resin purification (Qiagen) and eluted in a 20 μl volume. Automated sequencing reactions were performed by addition of 4 μl of template DNA to 8μl of ABI Prism BigDye Terminator Cycle Sequencing Ready Reaction mix (Perkin Elmer), 3.2 pmol primer and made to 20 μl volume with water. Standard cycle conditions were used for a Thermal Cycler Model 480 (Perkin Elmer), with reaction products ethanol precipitated, dried and submitted to an ABI 377 automated sequencer. Sequencing oligonucleotides spaced intermittently and in both directions throughout the coding region included forward hMSHR N-inner, Seq1 5′-TCTGACGGGCTCTTCCTC-3′ (SEQ ID NO: 43), Seq3 5′-TCCAGCCTCTGCTTCCTG-3′ (SEQ ID NO: 44), Seq4 5′-GCCCGGCTCCACAAGAGG-3′ (SEQ ID NO: 45) and reverse hMSHR C-inner, Seq5 5′-GCGCTGCCTCTTGTGGAG-3′ (SEQ ID NO: 46), Seq2 5′-ATGGAGCTGCAGGTGATC-3′ (SEQ ID NO: 47). To obtain the entire MC1R coding sequence it is usually sufficient to use hMSHR N-inner and Seq3, sequence anomalies or new MC1R variants can be checked by determination of the second strand using hMSHR C-inner and Seq5.

All of the family members who had contracted melanoma at the time of this analysis were genotyped by complete nucleotide sequencing as well as a total of 50 unaffected individuals selected from the pedigrees to determine MC1R variant content within the families. Automated sequencing of the MC1R coding region was performed by the addition of 100-200 ng of DNA template to 8 μL of ABI Prism dye terminator premix (utilizing AmpliTaq DNA polymerase FS [Perkin Elmer]), 3.2 pmol primer made up to 20 μL MilliQ water and covered with paraffin oil. Cycling was performed in a thermal cycler (Perkin Elmer model 480) using standard cycling conditions: 30 s 96° C., 15 s 50° C. and 4 min 60° C. for 25 cycles. PCR reaction products were ethanol precipitated, dried for 1 min at 95° C. and submitted to an ABI 373 automated sequencer. Sequence data were analyzed utilizing the Sequencher program (Genecodes).

The remaining family members were MC1R genotyped using a previously described ASO procedure (Palmer et al. 2000) using allele-specific oligonucleotides as set forth in Table 4. ASO Probes have been designed as 15 base oligonucleotides with the mismatch centrally positioned to provide maximal binding stability to the complementary sequence and instability of a mismatch.

Briefly, denatured inner PCR reaction product was blotted onto Magna (MSI) nylon transfer membrane and cross-linked under 150 mJ of UV energy. Following this, the blot was pre-hybridized in 10 mL of TMAC hybridization buffer; containing 3 M TMAC, 1 mM EDTA, 25 mM Na₃HPO₄ pH 6.8, 0.1% SDS, 5× Denhardt's solution and 0.1 mg/mL sonicated salmon sperm DNA for 1 hour at 42° C. After the addition of between 50-100 μL of the γ-³²P-radiolabelled probe, dot-blots were left to hybridize overnight at 42° C. Unbound probe was removed by washing for 20 min at 50° C. in 10 mL of TMAC wash buffer consisting of 3 M TMAC, 1 mM EDTA, 25 mM Na₃HPO₄ pH 6.8 and 0.1% SDS. Genotypes were visualized using autoradiography.

Statistical Methods

Survival analysis was carried out using the survival 5 package of Therneau (1999) running on R 1.1.1. If MC1R and CDKN2A genotype are the sole or overwhelming causes of melanoma in these families, then analyses including these variables will eliminate problems due to the correlated nature of the data (i.e. familial nature). However, if other familial factors are acting to increase risk, these may confound the detection of effects of MC 1 R. The present inventors therefore performed Cox proportional hazards model analyses stratifying on pedigree or nuclear family, and also including family as a clustering variable in a gamma frailty model, in an attempt to control for residual familial correlation in risk of melanoma. Stratifying on nuclear families, and restricting the analysis to siblings only is an equivalent to the sibling-transmission disequilibrium test (sib-TDT; Spielman & Ewens 1996) method of adjusting for population stratification. The Cox proportional hazards model was also used to generate the survival curve showing age-specific probability of developing melanoma for the Queensland population at large using all cases of invasive melanoma reported to the Queensland Cancer Registry in 1996.

Results

There were 15 familial melanoma pedigrees available to this study, in which 9 different CDKN2A mutations were segregating (Table 5). The families contained a total of 53 sibships and 270 individuals, with an average of 6.5 melanoma cases per kindred. The number of members for which DNA or genotype information was available is shown in Table 6. MC1R genotype was determined in 136 family members and CDKN2A genotype was available for 162 individuals, but only 131 of these individuals have both loci genotyped. There were 97 melanoma cases within the sample, 76 of whom were found to carry a CDKN2A mutation. Ten (12%) were designated as sporadic cases based upon absence of any CDKN2A mutation; this includes one case each in pedigrees 40599, 40750, 40823, 41031 and 41162, two cases in pedigree 41105 and three in 60001. CDKN2A mutation data were not available for 11 cases (Table 5). There was a total of 60 pedigree members with recorded skin color and 57 with hair color.

Allele frequencies for the eight MC1R variants detected in family members are shown in Table 7, together with frequency data from two samples of controls drawn from the general population (Palmer et al. 2000). The variant frequencies were similar to those observed in the south-east Queensland population except for the Arg151Cys variant which is present in around 26% of family members versus a general population frequency of approximately 10%. Only 14% of those genotyped did not carry an MC1R variant, and notably, all of the seven sporadic melanoma cases (CDKN2A mutation negative) for whom MC1R genotype was available carried variants. In order to maximize the information available for skin and hair color, association with MC1R variants was tested with simple grouping of all study members irrespective of pedigree or relationship. When all MC1R variants were grouped, there was a significant tendency for MC1R variants to increase in frequency as hair colors lightened and became red (Mantel-Haenszel test for trend P=0.01). This was more marked when the three previously defined RHC variants, Arg151Cys, Arg160Trp and Asp294His were pooled (P<0.001). A similar trend was observed with skin color, although all variants considered together were not significantly associated with skin color and the association with RHC variants was marginal (P=0.03). Carrying a CDKN2A mutation showed no association with skin or hair color in this dataset.

The unstratified survival analysis including MC1R and CDKN2A consensus homozygote versus one or more variants (Table 8) showed that carrying an MC1R variant allele significantly increased the risk of developing melanoma in both CDKN2A carriers and non-carriers (P=0.005). The impact on melanoma risk of carrying both an MC1R variant and a CDKN2A mutation is demonstrated by a significant increase in raw penetrance (shown as percent of melanoma cases) from 50% to 83.8% and a decrease in the mean age at onset from 58.1 to 37.8 years when compared to family members who carry a CDKN2A mutation alone (P=0.01). There was little difference in risk of melanoma between family members carrying one versus two variant MC1R alleles (OR=3.3, 95% CI 1.25-8.59; OR=3.4 95% CI 1.31-8.78, respectively; consensus genotype reference category).

Multiple tumors were common among cases within these families who carried a CDKN2A mutation (70%), but were seen in only one of seven CDKN2A mutation-negative cases. There was a trend for CDKN2A mutation negative cases carrying the MC1 R consensus genotype to have fewer tumors, but the numbers in this group (with information) are small (¼ versus 35/50).

The Cox proportional hazard method was also used to assess the influence of both CDKN2A mutation and MC1R variants on propensity to develop melanoma (Table 9). Family members carrying a CDKN2A mutation were 13.35-fold (95% CI 6.01-29.67) more likely to develop melanomas than those individuals who were wild type. Carriage of any MC1R variant significantly increased the likelihood of developing melanoma in these pedigrees 3.72-fold (95% CI 1.48-9.37). When stratifying on sex, the hazard ratios demonstrate an unusual tendency for more females than males to develop melanoma, although this was not significant. The analyses that included pedigree as a covariate obtained similar parameter estimates (MC1R OR=4.7, 95% CI 1.1-19.9) to those shown in Table 9, as did those conditioning on pedigree (MC1R OR=3.3, 95% CI 0.70-16.0), though the latter led to a loss of power reflected in a non-significant P-value. Stratifying on sibship reduced the sample size and broadened the confidence interval still further. The frailty models using pedigree and sibship as clustering variables estimated the frailty variance as not significantly different from zero, suggesting that there were no large residual familial correlations that might confound the estimates of the effects of CDKN2A and MC1R.

When the Cox proportional hazard analysis was restricted to either the Arg151Cys variant (the most common allele in these kindreds), or to the combination of the RHC variants, similar parameter estimates were obtained, but they were less significant than when all MC1R variants were pooled (for Arg151Cys, OR=1.48, 95% CI 0.91-2.42; for the RHC variants, OR=2.02, 95% CI 1.16-3.12). Grouping the remaining MC1R variants other than the three major variants important in hair and skin color determination gave a hazard ratio of 1.32 (Table 9), which was not significantly different from those individuals carrying a consensus MC1R genotype, indicating that most of the effect of MC1R genotype on melanoma risk is due to the Arg151Cys, Arg160Trp and Asp294His alleles. There were only 14 MC1R heterozygous parents of an affected child available for analysis, and the transmission disequilibrium test (TDT) analysis was not significant (9/14 variant alleles transmitted, P=0.42).

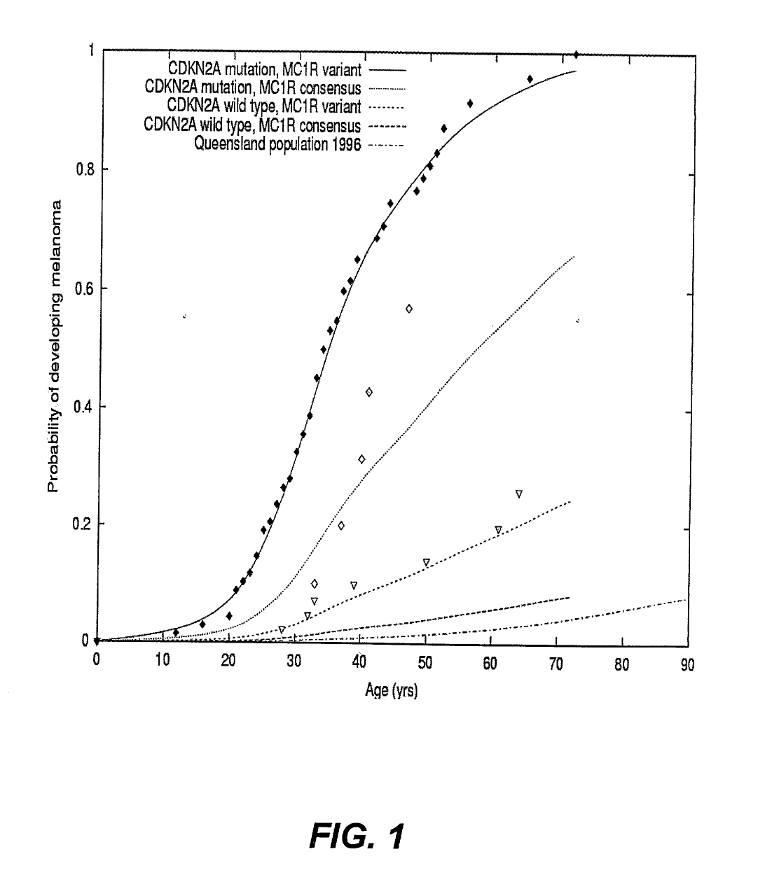

Since the present inventors detected no significant confounding effect of pedigree membership, we plotted predicted survival curves from the unstratified proportional hazards model analysis (FIG. 1). The survival curves plot the age-related probability of developing melanoma for each CDKN2A and MC1R genotype category for each of the four genotype categories presented in Table 8. Probability of developing melanoma is measured as the proportion of pedigree members with a given genotype who have developed a melanoma by a given age. The smoothed survival curves were calculated by pooling members from all pedigrees, the validity of which was confirmed through stratification on pedigree in the survival analysis. The age-specific probability of developing melanoma within the Queensland population at large is also shown in FIG. 1. Despite the small numbers in some covariate subgroups, the estimates for risk associated with the MC1R and CDKN2A “wild-type” genotype are close to those for the Queensland general population. This model assumes an absence of interaction, and proportional hazards.

### EXAMPLE 2

Family Material and Control Subjects

The complete pedigree data, method of ascertainment and histopathological findings on the six 9p-linked Dutch FAMMM syndrome families have been published elsewhere (Bergman et al. 1992). From these families, 101 carriers of a 19 basepair deletion within the p16 gene (p16-Leiden) were included in an SSCP analysis of MC1 using a technique based on that described in Orita et al. 1989.

Skin type was assessed by the skin type classification of Fitzpatrick & Breathnach 1963. In this classification Caucasian (white) skin is divided into three skin phenotypes. Skin type I represents a fair skin that always burns and never tans but frequently freckles in the sun; skin type I is accompanied by red hair. Skin type II is lightly pigmented, easy burning and yielding a light sun tan. Most individuals with skin type II have fair hair and blue eyes. Skin type III is still a Caucasian skin type, characterized by easy tanning and not readily burning in the sun. These individuals tend to have brown hair and brown eyes. Skin type IV never burns and tans always with ease, and is not regarded as a Caucasian skin type.

Detection of MC1R Variants

Genomic DNA from family members of the Dutch FAMMM families was isolated from peripheral blood leukocytes by routine methods (Miller et al. 1988). A specific PCR product of MC1R coding sequence (Genbank accession number X65634) was digested by either 2 U RsaI or MspI and was screened for mutations by SSCP analysis on a 6% polyacrylamide gel with 10% glycerol. The gels were run at room temperature for 6 hours at 26 Watt or 16 hours at 20 Watt for MspI and RsaI digests respectively. PCR reaction mixtures contained 60 mM Tris-HCl, pH 10.0; 2.0 mM MgCl₂; 15 mM (NH₄)₂SO₄; 100 μM each dGTP, dTTP, dATP, dCTP; 1 μl [α-³²P]dCTP (3,000 Ci mmol⁻¹), 500 ng of each PCR primer, 2 U AmpliTaq (Perkin Elmer Cetus); and 10% DMSO in a total volume of 100 μl. 50 ng genomic template DNA were added to a 10 μl reaction mixture. Samples were covered with mineral oil, denatured for 4 min at 92° C., and passed through 33 cycles of amplification, consisting of 50 s denaturation at 92° C., 50 s primer annealing at 60° C., 2 min elongation at 72° C. The amplifications were carried out in 0.5 ml tubes (Perkin Elmer). The DNA sequences of the primers were: F-5′-CAACGACT-CCTTCCTGCTTC-3′ (SEQ ID NO: 48) and R-5′-TGCCCAGCACACTTAAAGC-3′ (SEQ ID NO: 49), resulting in a 1018 bp PCR fragment.

Sequence Analysis

For sequence analysis, internal primers were used to amplify MC1R in two fragments, which were purified by the EasyPrep™ PCR Product Prep Kit (Pharmacia Biotech). Products were directly sequenced by the AmpliCycle™ Sequencing Kit (Perkin Elmer) with the amplification primers. Sequences of the internal primers were:

| | |
|---|---|
| IF-5′-ACCTGCAGCTCCAT-GCTGTC-3′ | (SEQ ID NO:50) |
| and | |
| IR-5′-GTCACGATGCTGTGGTAGC-3′. | (SEQ ID NO:51) |

Statistical Analysis

For comparison of allele frequencies in different groups Pearson's chi-square test was applied, or Fisher's exact test whenever appropriate. The effect of the number of MC1R variants in the genotype on melanoma risk was evaluated using logistic regression. The Mantel-Haenszel chi-square test was used to test for effect of the MC1R variant Arg151Cys on melanoma risk conditional on skin type.

Association between MC1R variants and melanoma in Dutch FAMMM kindreds

The present inventors studied the role of MC1R variants in six Dutch FAMMM families in which a 19 bp deletion in exon 2 of the p16 gene (p16-Leiden) is segregating (Gruis et al. 1995a). Mutation analysis of the entire MC1R coding sequence was performed in 101 p16-Leiden carriers from these families, of whom 38 had developed a melanoma. Allele frequencies for six MC1R variants detected in p16-Leiden carriers are presented in Table 10, together with the frequency data from a large Dutch control population. In general, frequencies of variants in the p16-Leiden carriers paralleled those in the control population except for the Arg151Cys variant which was present in around 14% of the family members versus a general population frequency of 5% (p<10⁻⁶), and for the variant Ile155Thr (p≈0.001). The excess of the Arg151Cys allele in the p16-Leiden carriers is mostly due to a strongly frequency in the melanoma patients (21 %) (Table 10).

Within the families the Arg151Cys allele was transmitted from heterozygous parents carrying p16-Leiden to affected offspring more often than expected by chance. Out of 12 p16-Leiden positive offspring with melanoma, 8 had received the Arg151Cys allele from their heterozygous parent, while out of 16 unaffected offspring (all carrying p16-Leiden) only 6 had inherited the Arg151Cys allele (p≈0.07, one sided).

When all MC1R variant alleles were considered jointly as a potential melanoma risk factor in p16-Leiden carriers, a similar pattern was observed. Overall, 38% of the p16-Leiden carriers developed melanoma. Table 11 shows a melanoma frequency of 55% in p16-Leiden carriers with two MC1R variants, compared to a melanoma frequency of 18% in MC1R wildtype p16-Leiden carriers. Logistic regression showed that the melanoma risk increases with the number of MC1R variants in the genotype (p≈0.01), with an odds ratio of 2.4 for each additional MC1R variant in the genotype. When this analyses was carried out not including subjects with Arg151Cys alleles, the odds ratio per MC1R variant was still increased (OR=1.8), but it did not differ significantly from 1.0 (p≈0.16). The age at onset for melanoma was very similar in all genotype groups, varying between 34 years in the group with one MC1R variant and 42 years in the group with two MC1R variants. The distribution of skin types in p16-Leiden carriers was determined and found not to differ significantly from the control population (Table 12). However, within the group of p16-Leiden carriers, a significant difference in the skin type distribution existed between subjects with and without melanoma (p≈0.02), with a preponderance of fair skin types among melanoma patients. Subsequently, the effect of the MC1R variant Arg151Cys on melanoma risk was evaluated, correcting for the existing relationship between Arg151Cys and fair skin, and for the increased melanoma risk in fair skin. A Mantel Haenszel test conditional on skin type yielded a chi-square of 2.78 (p≈0.05, one-sided), which supports, albeit with marginal significance, that the Arg151Cys variant independently contributes to melanoma risk.

Discussion

This study demonstrates a significant impact of MC1R genotype on penetrance of CDKN2A mutations in melanoma-dense pedigrees. Presence of an MC1R variant in addition to a CDKN2A mutation significantly increased the raw melanoma penetrance, decreasing age of onset by up to 20 years compared to individuals carrying a CDKN2A mutation alone. At 50 years of age, 81% of people with both a CDKN2A mutation and an MC1R variant had developed melanoma whereas only 57% of those carrying a CDKN2A mutation alone had developed melanoma. Presence of an MC1R variant alone was estimated to account for a risk of 14% by 50 years of age. Although there were no melanoma cases without either CDKN2A mutations or MC1R variants, the Cox proportional hazards model predicted an overall melanoma risk of about 10% by 70 years of age for this genotype, close to the Queensland population lifetime melanoma risk previously estimated at about 1 in 17. Penetrance of CDKN2A mutations without regard to MC1R status has been estimated at about 58% by age 80 in the UK, about 76% in North America, and approximately 92% by the same age in Australia. Presence of a substantially higher environmental UV level within Australia was suggested to account for this increase in penetrance of CDKN2A mutations.

Frailty variance estimates were not significantly different from zero when both pedigree and sibship were used as clustering variables, suggesting that the effects of CDKN2A and MC1R together may account for the majority of melanoma within these pedigrees. No significant differences in trends in individual pedigrees were observed in the survival analyses, suggesting that individual CDKN2A mutations in these families tend to have similar effects on melanoma penetrance, although power to detect such differences is low. At present, there is a dearth of studies that assess differences in melanoma penetrance in families carrying different CDKN2A mutations and it can only be assumed that each CDKN2A mutation is acting similarly to increase risk of melanoma. It is clear that future attempts to more accurately assess the individual and combined impact of CDKN2A mutations on melanoma incidence will require knowledge of MC1R genotype status, which is demonstrated here to act as a significant modifier of CDKN2A penetrance. Tumor types other than melanoma were not over-represented in this cohort of pedigrees (manuscript in preparation), although other cohorts of families have been shown to have a significant excess of pancreatic cancers (Goldstein et al. 1995; Vasen et al. 2000).

Unlike CDKN2A mutations, MC1R variants clearly do not show functional equivalence in influencing melanoma risk. The RHC variants, Arg151Cys, Arg160Trp and Asp294His account for much of the MC1R effect in increasing CDKN2A penetrance, with the remaining variants not significantly associated with melanoma risk. The same three RHC variants are those identified in association with fair skin, freckling, poor tanning capacity, melanoma and non-melanocytic skin cancer risk (Box et al. 1997; Smith et al. 1998; Flanagan et al. 2000; Palmer et al. 2000; Box et al. 2001). Limited information on pigmentation phenotype was available on family members, preventing a more thorough analysis of the interactions with MC1R variants, CDKN2A mutation, and melanoma.

It is an interesting parallel that presence of an MC1R variant shifts the age-specific CDKN2A mutation penetrance curve towards younger ages in a similar way to that observed for those families ascertained in a high UV environment. MC1R activity is crucial for an effective skin response to UV exposure, noted by visible tanning, and carrying a single RHC MC1R variant is enough to significantly diminish the skin's capacity to respond in a protective way to UV exposure (Flanagan et al. 2000; Healy et al. 2000). The data presented herein suggest that carrying two MC1R variants adds no further risk than carrying one such variant, although a larger sample may reveal a difference. It is clear from earlier studies that presence of a single RHC MC1R variant is enough to give a significant heterozygote effect on pigmentation and melanoma risk (Healy et al. 2000; Palmer et al. 2000) and may act here to shift the eumelanin/pheomelanin balance and increase the amount of pheomelanin produced by the skin. Pheomelanin and related metabolites have been shown to be mutagenic and cytotoxic (Harsanyi et al. 1980; Sturm 1998), suggesting a dual UV sensitivity where increased levels of this type of melanin within the skin may not only have a diminished UV protective capacity, but may actively promote generation of skin tumors. A single MC1R variant may be enough to exert a marked intrinsic and non-pigmentation related effect on propensity for melanocytic cellular transformation.

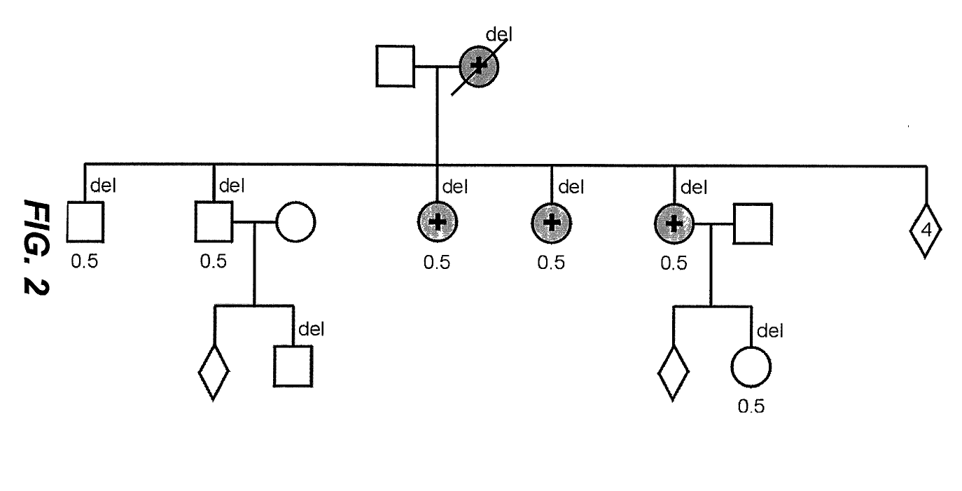

The effect of MC1R variant alleles on both sporadic melanoma and familial melanoma seems to be partly mediated via determination of pigmentation phenotype and the MC1R alleles involved, and seemingly negates the protection normally afforded by a darker skin color. While the sample size of the study in Example 2 is modest, the effects of various tentative melanoma risk factors can be evaluated more easily since they are measured in a sample of individuals who all carry the same p16 mutation. In Example 2, the present inventors have found an increased frequency of MC1R variants, in particular of variant Arg151Cys, in melanoma patients. Furthermore, they were able to show that within families the Arg151Cys allele was transmitted from heterozygous parents to more than 50% of melanoma-positive offspring, and to fewer than 50% of their melanoma-free offspring. This transmission distortion indicates that the increased frequency of Arg151Cys in melanoma patients is not simply a consequence of the relatively close family-relationships that exist between some of the individuals in the sample as illustrated in FIG. 2. While it appears that some of the increased risk in Arg151Cys positive p16-Leiden carriers can be attributed to the fair skin type that is associated with this variant, our stratified statistical analysis supports the notion that Arg151Cys also contributes an increased risk independently of its effect on skin type.

In conclusion, the present inventors have presented evidence that variant alleles at the MC1R locus significantly increase penetrance of mutations at the CDKN2A locus, and propose that this is one of the few good examples of gene-gene interaction on disease risk documented to date. One of the major goals for researchers attempting to understand the complexities of melanoma etiology is to identify host factors that influence age of onset, number of primary tumors, tumor site, and time to metastasis. Obtaining reliable predictors of risk and prognosis offers the promise of better management and prevention of many tumor types. This is nowhere more relevant than for melanoma which, if caught early, is eminently treatable but has very poor prognosis following metastasis.

Throughout this specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated herein by reference in its entirety.

Table 1. Examples of primer combinations used for amplification of CDKN2A genomic DNA and cDNA. *temperature range indicated upper and lower limits of the touchdown polymerase chain reaction (PCR) cycling conditions. P96F=SEQ ID NO: 1; P968R=SEQ ID NO: 2; P781F=SEQ ID NO: 3; P1424R=SEQ ID NO: 4; 42F=SEQ ID NO: 5; 551R=SEQ ID NO: 6; X3P2F=SEQ ID NO: 7; X3P2R=SEQ ID NO: 8; RT957F=SEQ ID NO: 9; 346R=SEQ ID NO: 10; 200F=SEQ ID NO: 11.

Table 2. Examples of primer combinations used for amplification of CDKN2A genomic DNA. These primer combinations produce shorter amplification products for single strand conformational polymorphism (SSCP) analysis. X1.31F=SEQ ID NO: 12; X1.26R=SEQ ID NO: 13; 2F=SEQ ID NO: 14; 1108R=SEQ ID NO: 15; X2.62F=SEQ ID NO: 16; 551R=SEQ ID NO: 6; X3P2F=SEQ ID NO: 7; X3P2R=SEQ ID NO: 8; 286R=SEQ ID NO: 17; 346R=SEQ ID NO: 10; 200F=SEQ ID NO: 11; 305F=SEQ ID NO: 18; X2.42R=SEQ ID NO: 19; X3.90F=SEQ ID NO: 20; 530R=SEQ ID NO: 21.

Table 3. Oligomers for allele specific oligonucleotide (ASO) hybridization analysis of CDKN2A (SEQ ID NOS: 22-37 in descending order). *mut=mutant allele; poly=polymorphic allele; wt=wildtype allele. § hyb. temp=hybridization temperatures; these were set at 7° C. below the Tm of the primer, calculated using the formula: Tm=4(G+C)+2(A+T).

Table 4. Oligomers for allele specific oligonucleotide (ASO) hybridization analysis of MC1R (SEQ ID NOS: 52-76 in descending order).

Table 5. Melanoma kindreds. ^{a}Pedigrees 40582 and 40823 each contain one spouse who had developed melanoma

Table 6. Genotyped family members available for analyses. ^{a}Not available; ^{b}Wild type for CDKN2A mutation at each of Gln50Arg, Arg24Pro, 46delC, Leu32Pro, Asp108Asn, Leu16Pro, Gly35Ala, 9del24, 33ins24, and Met53Ile positions; ^{c}Consensus genotype at each of the Val60Leu, Asp84Glu, Val92Met, Arg142His, Arg151Cys, Ile55Thr, Arg160Trp, Arg163Gln, and Asp294His positions.

Table 7. Frequencies of MC1R variants in 136 typed individuals. ^{a}MC1R variant allele frequency estimated in an unselected sample of 390 haplotypes from southeast Queensland presented in Palmer et al. 2000; ^{b}MC1R variant allele frequency estimated in a second unselected sample of 1627 haplotypes from southeast Queensland; ^{c}Not Available; ^{d}P<0.001.

Table 8. Mean disease-free survival by CDKN2A and MC1R genotype. ^{a}0 represents wild type CDKN2A or consensus MC1R at all positions analysed; ^{b}1 represents one or two MC1R variants; ^{c}All carried as 1=heterozygote wild type/mutant CDKN2A genotype; ^{d}P value for difference between CDKN2A=1 group with and without MC1R variant, P=0.01.

Table 9. Hazard ratios for CMM versus CDKN2A genotype, MC1R genotype and sex calculated using Cox proportional hazards method. ^{a}Each CDKN2A mutation is carried in heterozygote form; ^{b}members carrying 1 or 2 MC1R variants considered together in this analysis; red hair color (^{c}RHC) MC1R variants defined as Arg151Cys, Arg160Trp and Asp294His. ^{d}Other MC1R variant defined for this analysis as Val160Leu, Asp84Glu, Val92Met, Ile155Thr, and Arg163Gln.

Table 10: Allele frequencies of MC1R variants in 101 typed p16-Leiden carriers. a. allele frequencies of MC1R variants estimated in 385 control individuals from the Netherlands b. numbers are given in parenthesis.

Table 11: Association between MC1R variants and melanoma risk in 101 p16-Leiden carriers. a. non-Arg151Cys means MC1R variant other than Arg151Cys.

Table 12: Skin type distribution of the general Dutch population and p16-Leiden carriers. a. skin type was assessed for 385 individuals of the general Dutch population; b for 90 of the 101 p16-Leiden carriers data on skin type was available; c. skin type frequencies are given in parenthesis.

TABLE 1

| | | | | | |
|---|---|---|---|---|---|
| Amplified fragment | Primers | Sequence | SEQ ID | Annealing | Product |

| | | | | | |
|---|---|---|---|---|---|
| Genomic promoter | P96F | AAAGCAGGGGGCACTCATATTC | 1 | 65°-55° | 893 |
| fragment | P968R | TCCGAGCACTTAGCGAATGT | 2 | | |
| Genomic promoter | P781F | ACGCACTCAAACACGCCTTTG | 3 | 65°-59° | 642 |
| fragment | P1424R | CAAACTTCGTCCTCCAGAGTC | 4 | | |
| Exon 2 | 42F | GGAAATTGGAAACTGGAAGC | 5 | 60°-55° | 509 |
| | 551R | TCTGAGCTTTGGAAGCTCT | 6 | | |
| Exon 3 | X3P2F | GACGGCAAGAGAGGAGGG | 7 | 60°-55° | 206 |
| | X3P2R | AAAACTACGAAAGCGGGGTGG | 8 | | |
| RT-PCR promoter | RT957F | TTCAGGGGTGCCACATTC | 9 | 60°-55° | 844 |
| fragment | 346R | CCAGGTCCACGGGCAGA | 10 | | |
| RT-PCR 3′UTR | 200F | AGCCCAACTGCGCCGAC | 11 | 60°-55° | 777 |
| fragment | X3P2R | AAAACTACGAAAGCGGGGTGG | 8 | | |

TABLE 2

| Primer | | | | Tm | Product |
|---|---|---|---|---|---|
| Name | Exon | Primer Sequence (5′ to 3′ ) | Length | (°C.) | size |
| X1.31F | 1 | GGGAGCAGCATGGAGCCG (SEQ ID NO: 12) | 18 | 62 | 167 bp |
| X1.26R | | AGTCGCCCGCCATCCCCT (SEQ ID NO: 13) | 18 | 62 | |
| 2F | 1 | GAAGAAAGAGGAGGGGCTG (SEQ ID NO: 14) | 19 | 58 | |
| 1108R | | GCGCTACCTGATTCCAATTC (SEQ ID NO: 15) | 20 | 60 | 340 bp |
| 42F | 2 | GGAAATTGGAAACTGGAAGC (SEQ ID NO: 5) | 20 | 58 | 509 bp |
| 551R | | TCTGAGCTTTGGAAGCTCT (SEQ ID NO: 6) | 19 | 56 | |
| X2.62F | 2(a) | AGCTTCCTTTCCGTCATGC (SEQ ID NO: 16) | 19 | 58 | 203 bp |
| 286R | | GCAGCACCACCAGCGTG (SEQ ID NO: 17) | 17 | 58 | |
| 200F | 2(b) | AGCCCAACTGCGCCGAC (SEQ ID NO: 11) | 17 | 58 | 147 bp |
| 346R | | CCAGGTCCACGGGCAGA (SEQ ID NO: 10) | 17 | 58 | |
| 305F | 2(c) | TGGACGTGCGCGATGC (SEQ ID NO: 18) | 16 | 54 | 189 bp |
| X2.42R | | GGAAGCTCTCAGGGTACAAATTC (SEQ ID NO: 19) | 23 | 68 | |
| X3P2F | 3 | GACGGCAAGAGAGGAGGG (SEQ ID NO:7) | 18 | 60 | 206 bp |
| X3P2R | | AAAACTACGAAAGCGGGGTGG (SEQ ID NO: 8) | 21 | 64 | |
| X3.90F | 3 | CCGGTAGGGACGGCAAGAGA (SEQ ID NO: 20) | 20 | 66 | |
| 530R | | CTGTAGGACCTTCGGTGACTGATGA (SEQ ID NO: 21) | 25 | 76 | 125 bp |

TABLE 3

| | DKN2A | Oligomer sequence | SEQ ID | hyb. temp.§ |
|---|---|---|---|---|
| Oligomer specificity* | exon | (5′-3′) | NO: | (°C.) |
| R24P wt | 1 | CCGGGGTCGGGTAGAGG | 22 | 53 |
| R24P mut | 1 | CCGGGGTCCGGTAGAGG | 23 | 53 |
| L32P wt | 1 | GGCTGCTGGAGGCGGT | 24 | 49 |
| L32P mut | 1 | GGCTGCCGGAGGCGGT | 25 | 51 |
| M53I wt | 2 | AGGTCATGATGATGGGCA | 26 | 47 |
| M53I mut | 2 | AGGTCATGATCATGGGCA | 27 | 47 |
| G101W wt | 2 | ACCGGGCCGGGGCGCGG | 28 | 59 |
| G101W mut | 2 | ACCGGGCCTGGGCGCGG | 29 | 57 |
| V127D wt | 2 | TCGCGATGTCGCACGGT | 30 | 49 |
| V127D mut | 2 | TCGCGATGACGCACGGT | 31 | 49 |
| A148T wt | 2 | TAGATGCCGCGGAAGGT | 32 | 47 |
| A148T poly | 2 | TAGATGCCACGGAAGGT | 33 | 45 |
| Nt500 wt | 3 | GAAACCTCCGGAAACTTAG | 34 | 49 |
| Nt500 poly | 3 | GAAACCTCGGGAAACTTAG | 35 | 49 |
| Nt54O wt | 3 | TACAGGGCCACAACTGG | 36 | 41 |
| Nt540 poly | 3 | TACAGGGCTACAACTGG | 37 | 43 |

TABLE 4

| | | | |
|---|---|---|---|
| V60L | Val | gctggtggtggccac | SEQ ID NO: 52 |
| | Leu | gctggtgTtggccac | SEQ ID NO: 53 |
| K65N | Lys | tcgccaagaaccgga | SEQ ID NO: 54 |
| | Asn | tcgccaaTaaccgga | SEQ ID NO: 55 |
| D84E | Asp | tgtcggacctgctgg | SEQ ID NO: 56 |
| | Glu | tgtcggaActgctgg | SEQ ID NO: 57 |
| V92M | Val | gagcaacgtgctgga | SEQ ID NO: 58 |
| | Met | gagcaacAtgctgga | SEQ ID NO: 59 |
| V92L | Leu | gagcaacTtgctgga | SEQ ID NO: 60 |
| R142H | Arg | gtggaccgctacatc | SEQ ID NO: 61 |
| | His | gtggaccActacatc | SEQ ID NO: 62 |
| R151C | Arg | cgcactgcgctacca | SEQ ID NO: 63 |
| | Cys | cgcactgTgctacca | SEQ ID NO: 64 |
| I155L | Ile | cacagcatcgtgacc | SEQ ID NO: 65 |
| | Leu | cacagcaCcgtgacc | SEQ ID NO: 66 |
| R160W | Arg | cctgccgcgggcgcg | SEQ ID NO: 67 |
| | Trp | cctgccgTgggcgcg | SEQ ID NO: 68 |
| R163Q | Arg | gcgcggcgagccgtt | SEQ ID NO: 69 |
| | Gln | gcgcggcAagccgtt | SEQ ID NO: 70 |
| D294H | Asp | catcatcgaccccct | SEQ ID NO: 71 |
| | His | catcatcCaccccct | SEQ ID NO: 72 |
| A299T | Ala | catctacgccttcca | SEQ ID NO: 73 |
| | Thr | catctacAccttcca | SEQ ID NO: 74 |
| 537insC | wt | gctcttcatcgccta | SEQ ID NO: 75 |
| | insC | gctcttcCatcgcct | SEQ ID NO: 76 |

TABLE 5

| Pedigree | Number of | CDKN2A | Number of | Number | Number CMM | Number of |
|---|---|---|---|---|---|---|
| ID | members | mutation | CMM cases | unaffected | status unknown | Spouses |
| 40300 | 17 | R24P | 8 | 7 | 2 | 3 |
| 40582 | 24 | 33ins24 | 6 | 16 | 2 | 3^{a} |
| 40599 | 30 | Q50R | 12 | 17 | 1 | 4 |
| 40750 | 20 | G35A | 5 | 15 | 0 | 2 |
| 40787 | 18 | 46delC | 6 | 12 | 0 | 3 |
| 40823 | 14 | L32P | 6 | 8 | 0 | 2^{a} |
| 40935 | 10 | L16P | 3 | 5 | 0 | 1 |
| 41001 | 27 | M53I | 11 | 16 | 0 | 5 |
| 41019 | 8 | R24P | 2 | 6 | 0 | 1 |
| 41031 | 19 | M53I | 5 | 14 | 0 | 2 |
| 41105 | 23 | D108N | 4 | 19 | 0 | 2 |
| 41119 | 12 | 33ins24 | 7 | 5 | 0 | 2 |
| 41156 | 4 | M53I | 2 | 0 | 2 | 2 |
| 41162 | 8 | M531 | 3 | 1 | 4 | 0 |
| 60001 | 38 | M53I | 17 | 21 | 0 | 7 |
| Totals | 270 | | 97 | 162 | 11 | 39 |

TABLE 6

| | | Genotypic | | CMM status | |
|---|---|---|---|---|---|
| locus | | Data | Affected | Unaffected | Unknown |
| CDKN2A | N/A^{a} | 108 | 11 | 86 | 11 |
| | wild type^{b} | 68 | 10 | 58 | 0 |
| | mutant | 94 | 76 | 18 | 0 |
| MC1R | N/A | 134 | 30 | 93 | 11 |
| | consensus^{c} | 20 | 5 | 15 | 0 |
| | 1 variant | 58 | 28 | 30 | 0 |
| | 2 variants | 58 | 34 | 24 | 0 |

TABLE 7

| | | Population | Population |
|---|---|---|---|
| | Melanoma | Sample 1^{a} | Sample 2^{b} |
| | Family | Allele | Allele |
| | Allele | Frequency1 | Frequency2 |
| Variant | Frequency | (n = 390) | (n = 1627) |
| V60L | 0.109 | 0.120 | 0.124 |
| D84E | 0.011 | 0.015 | 0.011 |
| V92M | 0.109 | N/A^{c} | 0.097 |
| R151C^{d} | 0.255 | 0.089 | 0.111 |
| I155T | 0.007 | N/A | 0.010 |
| R160W | 0.084 | 0.076 | 0.071 |
| R163Q | 0.051 | N/A | 0.050 |
| D294H | 0.011 | 0.031 | 0.028 |

TABLE 8

| | | CMM cases | Mean age- | |
|---|---|---|---|---|
| Genotype | Number | (percent) | at-onset | SE |
| CDKN2A = 0, | 10 | 0 (0.0) | — | — |
| MC1R = 0^{a} | | | | |
| CDKN2A = 0, | 45 | 7 (15.6) | 82.4 | 3.90 |
| MC1R = 1^{b} | | | | |
| CDKN2A = 1^{c}, | 10 | 5 (50.0) | 58.1 | 7.23 |
| MC1R = 0 | | | | |
| CDKN2A = 1, | 68 | 57 (83.8) | 37.8 | 1.75 |
| MC1R = 1^{d} | | | | |

TABLE 9

| Risk factor | Hazard Ratio | lower 95% CI | upper 95% CI |
|---|---|---|---|
| CDKN2A mutant^{a} | 13.35 | 6.01 | 29.67 |
| MC1R variant^{b} | 3.72 | 1.48 | 9.37 |
| RHC^{c} variant | 2.02 | 1.16 | 3.12 |
| other^{d} variant | 1.32 | 0.88 | 1.98 |
| Female sex | 1.48 | 0.92 | 2.40 |

TABLE 10

| | | p16-Leiden | | General |
|---|---|---|---|---|
| | p16-Leiden | non- | p16-Leiden | popu- |
| | carriers | melanoma | melanoma | lation^{a} |
| Variant | (n = 101) | (n = 63) | (n = 38) | (n = 385) |
| V60L | 0.074 (15)^{b} | 0.071 (9) | 0.079 (6) | 0.083 (64) |
| V92M | 0.089 (18) | 0.095 (12) | 0.079 (6) | 0.069 (53) |
| R151C | 0.139 (28) | 0.095 (12) | 0.211 (16) | 0.048 (37) |
| I155T | 0.035 (7) | 0.016 (2) | 0.066 (5) | 0.003 (3) |
| R160W | 0.149 (30) | 0.127 (16) | 0.182 (14) | 0.105 (81) |
| R163Q | 0.069 (14) | 0.087 (11) | 0.039 (3) | 0.049 (38) |

TABLE 11

| | | melanoma |
|---|---|---|
| | | cases |
| genotype | total | (fraction) |
| p16-Leiden, MC1R = Wt/Wt | 17 | 3 (0.18) |
| p16-Leiden, MC1R = Wt/Var | 55 | 19 (0.35) |
| p16-Leiden, MC1R = Wt/non | 45 | 14 (0.31) |
| Arg151Cys | | |
| p16-Leiden, MC1R = Var/Var | 29 | 16 (0.55) |
| p16-Leiden, MC1R = non | 12 | 5 (0.42) |
| Arg151Cys/non Arg151Cys^{a} | | |

TABLE 12

| | General | p16-Leiden | p16-Leiden | p16-Leiden |
|---|---|---|---|---|
| Skin | population | carriers | non-melanoma | melanoma |
| type | (n = 385)^{a} | (n = 90)^{b} | (n = 58) | (n = 32) |
| I | 25 (0.07)^{c} | 10 (0.11) | 3 (0.05) | 7 (0.22) |
| II | 155 (0.40) | 38 (0.42) | 23 (0.40) | 15 (0.47) |
| III | 181 (0.47) | 42 (0.47) | 32 (0.55) | 10 (0.31) |
| IV | 24 (0.06) | — | — | — |

### REFERENCES

Aitken J, Welch J, Duffy D, Milligan A, Green A, Martin N, Hayward N (1999) CDKN2A variants in a population-based sample of Queensland families with melanoma. J Natl Cancer Inst 91:446-452

Aitken J F, Green A C, MacLennan R, Youl P, Martin N G (1996) The Queensland Familial Melanoma Project: study design and characteristics of participants. Melanoma Res 6:155-165

Bergman W, Gruis N A, Frants R R (1992) The Dutch FAMMM family material: clinical and genetic data. Cytogenet Cell Genet 59:161-4

Bliss J M, Ford D, Swerdlow A J, Armstrong B K, Cristofolini M, Elwood J M, Green A, et al (1995) Risk of cutaneous melanoma associated with pigmentation characteristics and freckling: systematic overview of 10 case-control studies. The International Melanoma Analysis Group (IMAGE). Int J Cancer 62:367-376

Box N F, Duffy D L, Irving R E, Russell A, Chen W, Griffiths L R, Parsons P G, et al (2001) Melanocortin-1 Receptor Genotype is a Risk Factor for Basal and Squamous Cell Carcinoma. Journal of Investigative Dermatology 116:224-229

Box N F, Wyeth J R, O'Gorman L E, Martin N G, Sturm R A (1997) Characterization of melanocyte stimulating hormone receptor variant alleles in twins with red hair. Hum Mol Genet 6:1891-1897

Breitbart M, Garbe C, Buttner P, Weiss J, Soyer H P, Stocker U, Kruger S, et al (1997) Ultraviolet light exposure, pigmentary traits and the development of melanocytic naevi and cutaneous melanoma. A case-control study of the German Central Malignant Melanoma Registry. Acta Derm Venereol 77:374-378

Castellano M, Pollock P M, Walters M K, Sparrow L E, Down L M, Gabrielli B G, Parsons P G and Hayward N K (1997) CDKN2A/p16 is inactivated in most melaonma cell lines. Cancer Res. 57:4868-75

Dutton C M, Paynton C, Sommer S S (1993) General method for amplifying regions of very high G+C content. Nucleic Acids Res 21:2953-2954

Flanagan N, Healy E, Ray A, Philips S, Todd C, Jackson I J, Birch-Machin M A, et al (2000) Pleiotropic effects of the melanocortin 1 receptor (MC1R) gene on human pigmentation. Hum Mol Genet 9:2531-2537

Flores J F, Pollock P M, Walker G J, Glendening J M, Lin A H, Palmer J M, Walters M K, et al (1997) Analysis of the CDKN2A, CDKN2B and CDK4 genes in 48 Australian melanoma kindreds. Oncogene 15:2999-3005

Fodde R. & Losekoot M. (1994) Mutation detection by denaturing gradient gel electrophoresis (DGGE). Hum. Mutat. 3:83-93

Garbe C, Buttner P, Weiss J, Soyer H P, Stocker U, Kruger S, Roser M, et al (1994) Risk factors for developing cutaneous melanoma and criteria for identifying persons at risk: multicenter case-control study of the Central Malignant Melanoma Registry of the German Dermatological Society. J Invest Dermatol 102:695-699

Gilchrest B A, Park H Y, Eller M S, Yaar M (1996) Mechanisms of ultraviolet light-induced pigmentation. Photochem Photobiol 63:1 -10

Goldstein A M, Fraser M C, Struewing J P, Hussussian C J, Ranade K, Zametkin D P, Fontaine L S, et al (1995) Increased risk of pancreatic cancer in melanoma-prone kindreds with p16INK4 mutations. N Engl J Med 333:970-974

Grange F, Chompret A, Guilloud-Bataille M, Guillaume J C, Margulis A, Prade M, Demenais F, et al (1995) Comparison between familial and nonfamilial melanoma in France. Arch Dermatol 131:1154-1159

Gruis N A, Sandkuijl L A, van der Velden P A, Bergman W, Frants R R (1995a) CDKN2 explains part of the clinical phenotype in Dutch familial atypical multiple-mole melanoma (FAMMM) syndrome families. Melanoma Res 5:169-177

Grulich A E, Bataille V, Swerdlow A J, Newton-Bishop J A, Cuzick J, Hersey P, McCarthy W H (1996) Naevi and pigmentary characteristics as risk factors for melanoma in a high-risk population: a case-control study in New South Wales, Australia. Int J Cancer 67:485-91

Harding R M, Healy E, Ray A J, Ellis N S, Flanagan N, Todd C, Dixon C, Sajantila A, Jackson I J, Birch-Machin M A, Rees J L, (2000). Evidence for variable selective pressures at MC1R. Am. J. Hum. Genet. 66:1351-61

Harsanyi Z P, Post P W, Brinkmann J P, Chedekel M R, Deibel R M (1980) Mutagenicity of melanin from human red hair. Experientia 36:291-292

Hayward N K (1998) Melanoma susceptibility: Population-based incidence of germline CDKN2A mutations in selected families with cutaneous melanoma. Curr. Prac. Med. 1:47-49

Hayward N K (1999) Molecular pathology of cutaneous melanoma. In: Srivastava S, Henson D E, Gazder A (eds) Molecular pathology of early cancer. IOS Press, Amsterdam, Berlin, Oxford, Tokyo, Washington DC, pp 207-231

Healy E, Todd C, Jackson I J, Birch-Machin M, Rees J L (1999) Skin type, melanoma, and melanocortin 1 receptor variants. J Invest Dermatol 112:512-3

Healy E, Flannagan N, Ray A, Todd C, Jackson I J, Matthews J N, Birch-Machin M A, et al (2000) Melanocortin-1-receptor gene and sun sensitivity in individuals without red hair. Lancet 355:1072-1073

Hussussian C J, Struewing J P, Goldstein A M, Higgins P A, Ally D S, Sheahan M D, Clark W H, Jr., et al (1994) Germline p16 mutations in familial melanoma. Nat Genet 8:15-21

Kamb A, Gruis N A, Weaver-Feldhaus J, Liu Q, Harshman K, Tavtigian S V, Stockert E, et al (1994) A cell cycle regulator potentially involved in genesis of many tumor. Science 264:436-440

Kamb A, Shattuck-Eidens D, Eeles R, Liu Q, Gruis N A, Ding W, Hussey C, et al (1994) Analysis of the p16 gene (CDKN2) as a candidate for the chromosome 9p melanoma susceptibility locus. Nat Genet 8:23-26

Liu Q, Thorland E C, Heit J A, Sommer S S (1997) Overlapping PCR for bidirectional PCR amplification of specific alleles: a rapid one tube method for simultaneously differentiating homozygotes and heterozygotes. Genome Res. 7 389-399.

Lukas J, Parry D, Aagaard L, Mann D J, Bartkova J, Strauss M, Peters G, et al (1995) Retinoblastoma-protein-dependent cell-cycle inhibition by the tumour suppressor p16. Nature 375:503-506

Miller S A, Dykes D D, Polesky H F (1988) A simple salting out procedure for extracting DNA from human nucleated cells. Nucleic Acids Res 16:1215

Nobori T, Miura K, Wu D J, Lois A, Takabayashi K, Carson D A (1994) Deletions of the cyclin-dependent kinase-4 inhibitor gene in multiple human cancers. Nature 368:753-756

Orita M, Iwahana H, Kanazawa H, Hayashi K, Sekiya T (1989) Detection of polymorphisms of human DNA by gel electrophoresis as single-strand conformation polymorphisms. Proc Natl Acad Sci USA 86:2766-70

Palmer J S, Duffy D L, Box N F, Aitken J F, O'Gorman L E, Green A C, Hayward N K, et al (2000) Melanocortin-1 receptor polymorphisms and risk of melanoma: is the association explained solely by pigmentation phenotype? Am J Hum Genet 66:176-186

Pollock P M, Stark M S, Palmer J M, Walters M K, Aitken J F, Martin, N G and Hayward N K (2001). Mutation analysis of the CDKN2A promoter in Australian melanoma families. Genes Chromosomes Cancer 32:89-94.

Rana B K, Hewitt-Emmett D, Jin L, Chang B H, Sambuughin N, Lin M, Watkins S, Bamshad M, Jorde L B, Ramsay M, Jenkins T, Li W H, (1999). High polymorphism at the human melanocortin 1 receptor locus. Genetics 151:1547-57.

Ruas M, Peters G (1998) The p16INK4a/CDKN2A tumor suppressor and its relatives. Biochim Biophys Acta 1378:F115-177

Serrano M, Hannon G J, Beach D (1993) A new regulatory motif in cell-cycle control causing specific inhibition of cyclin D/CDK4. Nature 366:704-707

Smith R, Healy E, Siddiqui S, Flanagan N, Steijlen P M, Rosdahl I, Jacques J P, et al (1998) Melanocortin 1 receptor variants in an Irish population. J Invest Dermatol 111:119-122

Sooknanan et al. (1994) Fidelity of nucleic acid amplification with avian myeloblastoma virus reverse transcriptase and T7 RNA polymerase. Biotechniques 17: 1077-80

Soufir N, Avril M F, Chompret A, Demenais F, Bombled J, Spatz A, Stoppa-Lyonnet D, et al (1998) Prevalence of p16 and CDK4 germline mutations in 48 melanoma-prone families in France. The French Familial Melanoma Study Group. Hum Mol Genet 7:209-216

Spielman R S, Ewens W J (1996) The TDT and other family-based tests for linkage disequilibrium and association. Am J Hum Genet 59:983-989

Sturm R A (1998) Human pigmentation genes and their response to solar UV radiation. Mutat Res 422:69-76

Sturm R A, Box N F, Ramsay M (1998) Human pigmentation genetics: the difference is only skin deep. Bioessays 20:712-721

Suzukie I, Cone R D, Im S, Nordlund J, Abdel-Malek, Z A (1996). Binding of melanotropic hormones to the melanocortin receptor MC1R on human melanocytes stimulates proliferation and melanogenesis. Endocrinology 137:1627-33

Suzuki I, Im S, Tada A, Scott C, Akcali C, Davis M B, Barsh G, et al (1999) Participation of the melanocortin-1 receptor in the UV control of pigmentation. J Investig Dermatol Symp Proc 4:29-34

Therneau (1999) R Survival 5 software. Mayo Clinic Rochester.

Thody A J, Graham A (1998) Does alpha-MSH have a role in regulating skin pigmentation in humans? Pigment Cell Res 11:265-274

Tsatmalia M, Yukitake J, Thody A J (1999a) ACTH-17 is a more potent agonist at the human MC1 receptor than alpha-MSH. Cell. Mol. Biol. 45:1029-1034.

Tsatmalia M, Wakamatsu K, Graham A J, Thody A J (1999b) Skin POMC peptides. Their binding affinities and activation of the human MC1 receptor. Ann. NY Acad. Sci. 885:466-469.

Tyagi et al. (1996) Extremely sensitive, background-free gene detection using binary probes and beta replicase. Proc. Natl. Acad. Sci. USA 93:5395-400

Valverde P, Healy E, Jackson I, Rees J L, Thody A J (1995) Variants of the melanocyte-stimulating hormone receptor gene are associated with red hair and fair skin in humans. Nat Genet 11:328-330

Vasen H F, Gruis N A, Frants R R, van Der Velden P A, Hille E T, Bergman W (2000) Risk of developing pancreatic cancer in families with familial atypical multiple mole melanoma associated with a specific 19 deletion of p16 (p16-Leiden). Int J Cancer 87:809-811

Walker G J, Hussussian C J, Flores J F, Glendening J M, Haluska F G, Dracopoli N C, Hayward N K, et al (1995) Mutations of the CDKN2/p16INK4 gene in Australian melanoma kindreds. Hum Mol Genet 4:1845-1852

Whiteman D C, Milligan A, Welch J, Green A C, Hayward N K (1997) Germline CDKN2A mutations in childhood melanoma. J Natl Cancer Inst 89:1460

Whiteman D C, Parsons P G, Green A C (1998) p53 expression and risk factors for cutaneous melanoma: a case-control study. Int J Cancer 77:843-848

Zhu G, Duffy D L, Eldridge A, Grace M, Mayne C, O'Gorman L, Aitken J F, et al (1999) A major quantitative-trait locus for mole density is linked to the familial melanoma gene CDKN2A: a maximum-likelihood combined linkage and association analysis in twins and their sibs. Am J Hum Genet 65:483-492

Zuo L, Weger J, Yang Q, Goldstein A M, Tucker M A, Walker G J, Hayward N, et al (1996) Germline mutations in the p16INK4a binding domain of CDK4 in familial melanoma. Nat Genet 12:97-99

What is claimed is:

1. A method of identifying a predisposition to melanoma, said method including the step of determining whether an individual carrying a CDKN2A mutant allele also carries an MC1R variant allele, a presence of said variant MC1R allele indicating a predisposition of said individual to melanoma greater than that expected as a consequence of said CDKN2A mutant allele alone.

2. A method of identifying a predisposition to melanoma, said method including the step of determining whether an individual carrying a MC1R variant allele also carries a CDKN2A mutant allele, a presence of said CDKN2A mutant allele indicating a predisposition of said individual to melanoma greater than that expected as a consequence of said MC1R variant allele alone.

3. A method of identifying a predisposition of to melanoma, said method including the step of determining whether an individual carries an MC1R variant allele and a CDKN2A mutant allele, a presence of said CDKN2A mutant allele and said MC1R variant allele indicating a predisposition of said individual to melanoma greater than that expected as a consequence of said CDKN2A mutant allele or said MC1R variant allele alone.

4. The method of any one of claims 1, 2 or 3, wherein the CDKN2A mutant allele is selected from the group consisting of: Gln50Arg; Arg24Pro; 46delC; Leu32Pro; Asp108Asn; Leu16Pro; Gly35Ala; 9del24; 33ins24; p16-Leiden and Met53Ile.

5. The method of any one of claims 1, 2 or 3 wherein the MC1R variant allele is selected from the group consisting of: Val60Leu; Asp84Glu; Val92Met; Arg142His; Arg151Cys; Ile155Thr; Arg160Trp; Arg163Gln; and Asp294His.

6. The method of any one of claims 1, 2 or 3 wherein the MC1R variant allele is selected from the group consisting of: Arg151Cys; Arg160Trp; and Asp294His.

7. The method of any one of claims 1, 2 or 3 wherein when the MC1R variant allele is Arg151Cys and the CDKN2A mutant allele is not p16-Leiden.

8. The method of any one of claims 1, 2, or 3 wherein the MC1R variant allele and/or the CDKN2A mutant allele is amplified by a nucleic acid sequence amplification technique.

9. The method of claim 8, wherein the nucleic acid sequence amplification technique is PCR.

10. The method of claim 9 wherein the MC1R variant allele and/or the CDKN2A mutant allele are amplified from human blood DNA.

11. The method of claim 8, wherein the MC1R variant allele and/or the CDKN2A mutant allele is/are detected by a technique selected from the group consisting of:
(i) single stranded conformational polymorphism (SSCP);
(ii) allele-specific oligonucleotide (ASO hybridization; and
(iii) DNA sequencing.

12. A kit for identifying a predisposition to melanoma, said kit comprising one or more MC1R gene-specific primers and/or one or more CDKN2A gene-specific primers.

13. The kit of claim 12, further comprising one or more MC1R variant allele-specific probes and/or one or more CDKN2A mutant allele-specific probes.

14. The kit of claim 12, wherein the MC1R gene-specific primers are selected from the group consisting of: SEQ ID NO: 39; SEQ ID NO: 40; SEQ ID NO: 41; SEQ ID NO: 42; SEQ ID NO: 48 and SEQ ID NO: 49.

15. The kit of claim 12, wherein the CDKN2A gene-specific primers are selected from the group consisting of SEQ ID NOS: 1-21.

16. The kit of claim 13, wherein the MC1R variant allele-specific probes are selected from the group consisting of SEQ ID NOS: 52-76.

17. The kit of claim 13, wherein the CDKN2A mutant allele-specific probes are selected from the group consisting of SEQ ID NOS: 22-37.

18. The kit of claim 12 further including one or more MC1R gene-specific sequencing primers and/or one or more CDKN2A gene-specific sequencing primers.

19. The kit of claim 18 wherein the one or more MC1R sequencing primers are selected from the group consisting of SEQ ID NOS: 43-47.

20. The kit of claim 18 wherein the one or more CDKN2A sequencing primers are selected from the group consisting of SEQ ID NOS: 1-21 and SEQ ID NO: 38.
